(19) United States
(12) Patent Application Publication
(10) Pub. No.: US 2025/0116673 A1
(43) Pub. Date: Apr. 10, 2025

(54) METHODS TO DETERMINE CD4+ MEMORY T CELL RESPONSES TO SARS-COV-2 INFECTION OR VACCINATION

(71) Applicant: ST. JUDE CHILDREN'S RESEARCH HOSPITAL, INC., Memphis, TN (US)

(72) Inventors: Paul THOMAS, Memphis, TN (US); Mikhail POGORELYY, Memphis, TN (US); Anastasia MINERVINA, Memphis, TN (US)

(21) Appl. No.: 18/681,966

(22) PCT Filed: Aug. 10, 2022
(86) PCT No.: PCT/US2022/039932

§ 371 Date: Feb. 7, 2024

Related U.S. Application Data
(60) Provisional application No. 63/232,334, filed on Aug. 12, 2021.

(51) Int. Cl. G01N 33/68; G01N 33/50

(52) U.S. Cl. CPC G01N 33/6872; G01N 33/5041; G01N 33/505; G01N 2333/7051; G01N 2800/52

(57) ABSTRACT

Disclosed are methods for identifying or determining whether a subject exhibits a CD4⁺ memory T cell response to SARS-CoV-2 infection or vaccination, assessing the efficacy of a SARS-CoV-2 vaccine, and developing personalized SARS-CoV-2 treatment plans by detecting the presence and/or quantity of a particular T cell receptor a chain that recognizes a specific Spike protein epitope.

### INTRODUCTION

This application claims benefit of priority to U.S. Provisional Patent Application Ser. No. 63/232,334, filed Aug. 12, 2021, the content of which is incorporated herein by reference in its entirety.

This invention was made with government support under grant no. AI136514 awarded by the National Institutes of Health. The government has certain rights in this invention.

### BACKGROUND

The COVID-19 pandemic necessitated rapid late-stage clinical trials of mRNA vaccine technology. The two mRNA vaccines developed by Pfizer/BioNTech (BNT162b2) and Moderna (mRNA-1273) have proven instrumental in the initiation of widespread vaccination campaigns in the United States. Both vaccines engender very high-titer circulating Severe Acute Respiratory Syndrome Coronavirus 2 (SARS-CoV-2) Spike protein-specific antibody titers that neutralize the SARS-CoV-2 strain they were developed to fight, as well as other SARS-CoV-2 variants that have developed since the vaccine design phase. The mRNA vaccines exhibit the highest efficacy in phase 3 studies among widely used COVID-19 vaccines worldwide. Understanding exactly how mRNA vaccines elicit such robust and protective immune responses in humans is critical to the extension and application of this technology to develop novel vaccines against other important pathogens.

Neutralizing antibodies induced by mRNA vaccines appear to be the key correlate of protection from COVID-19 in animal models. The lymph node germinal center reaction is critical to the development of high-affinity antibodies and long-lived plasma cells. CD4⁺ T cell responses, including T follicular helper (T_{FH}) cell responses are necessary for the formation of germinal centers and critical to the development of both plasma cells and memory B cells in the lymph node, while other types of CD4⁺ cells (Th1) orchestrate the cellular antiviral response While measuring the antibody responses after vaccination or SARS-CoV-2 infection is a standard clinical procedure, accessing memory T cell responses is much more challenging. T cells recognize peptide antigens presented on the HLA molecules, encoded by the most polymorphic locus in human population. Determining the quality and quantity of the memory CD4⁺ T cells specific to immunodominant SARS-CoV-2 epitopes, presented on the common HLA alleles, can help to understand the role of these responses in developing potent T cell-dependent human B cell responses.

### SUMMARY OF THE INVENTION

This invention is based on the discovery that COVID-19 vaccines engender large numbers of Spike-specific T cells in the peripheral blood of vaccinated human patients, which can be detected by the presence of a particular T cell receptor α-chain sequence motif. Accordingly, this invention provides methods for determining whether a subject exhibits a CD4⁺ memory T cell response to SARS-CoV-2 infection or vaccination; determining efficacy of a SARS-CoV-2 vaccine; and developing a personalized SARS-CoV-2 treatment plan for a subject by detecting, in a population of immune cells from a subject, the presence or quantity of a T cell receptor α motif having the amino acid sequence Cys-Ala-Xaa₁-Xaa₂-Asn-Tyr-Gly-Gly-Ser-Gln-Gly-Asn-Leu-Ile-Phe (SEQ ID NO:2), wherein X₁ is Gly, Ala, or Val, and X₂ is any amino acid residue, wherein the presence or quantity of the T cell receptor α motif. In some aspects, the subject has at least a DPB1*04:01 or DPB1*04:02 HLA allele.

### DETAILED DESCRIPTION OF THE INVENTION

COVID-19 mRNA vaccines generate high concentrations of circulating anti-Spike antibodies and Spike-specific CD4⁺ T cells following prime-boost vaccination. Analysis of the T cell receptor repertoires of subjects post SARS-CoV-2 infection and post vaccination showed that a SARS-CoV-2 Spike peptide having the core sequence YVSQPFLMD (SEQ ID NO: 1) is presented by the DPB1*04:01/04:02 human leukocyte antigen (HLA) allele, which is the most frequent allele in the human population, and elicits a robust CD4 response in all DPB1*04:01/04:02-positive individuals. Moreover, this T cell response is characterized by a highly conserved motif in the α chain of responding T cell receptors (TCRs): TRAV35-CA[G/A/V]XNYGGSQGNLIF-TRAJ42 (SEQ ID NO: 2). Thus, the presence of this single α chain motif expanded in a subject's T cells is of use in assessing CD4⁺ T cell memory responses in more than half of the worldwide population. Thus, unlike other conventional tests for T cell immunity, which fail to take into account HLA-diversity, the present method provides the advantage of being of particular use in subjects having a specified HLA allele, i.e., DPB1*04:01/04:02.

Accordingly, the present invention provides methods for identifying or determining whether a subject exhibits a CD4⁺ memory T cell response to SARS-CoV-2 infection or vaccination, assessing the efficacy of a SARS-CoV-2 vaccine, and developing personalized SARS-CoV-2 treatment plans by detecting the presence and/or quantity of the TCRα chain sequence of SEQ ID NO:2 in said subjects. Such methods are of use in, e.g., detecting responses to SARS-CoV-2 vaccination or infection in immunocompromised patients with impaired antibody responses; providing information about longevity of cellular memory responses after natural infection and vaccination; providing a means to compare the potency of different vaccines to elicit CD4⁺ T cell responses; and detecting a current or recent COVID-19 infection.

“CD4” molecules are differentiation antigens present on T-lymphocytes, but also found on other cells including monocytes/macrophages. CD4 proteins are members of the immunoglobulin supergene family and are implicated as MHC associative recognition elements in (Major Histocompatibility Complex) Class II-restricted immune responses.

As used herein, the term “CD4⁺ memory T cell” or “memory T cell” has its general meaning in the art and refers a cell which expresses CD4 on the cell surface, is generated in the primary immune response, and can induce an effective and rapid secondary immune response to previously encountered pathogens or antigens. Thus, a “CD4⁺ memory T cell response” refers to the generation or presence of a CD4⁺ memory T cell upon exposure to a pathogen or antigen. In some aspects, the memory T cell response is a memory T follicular helper (T_{FH}) cell response. T_{FH} cells are the subset of CD4⁺ T helper cells that are required for generation and maintenance of germinal center reactions and the generation of long-lived humoral immunity. This specialized T helper subset provides help to cognate B cells via their expression of CD40 ligand, IL-21, IL-4, and other molecules. T_{FH} cells are characterized by their expression of the chemokine receptor CXCR5, expression of the transcriptional repressor Bcl6, and their capacity to migrate to the follicle and promote germinal center B cell responses.

Subjects benefiting from the methods of this invention include subjects exposed to the SARS-CoV-2 virus, subjects who have had a SARS-CoV-2 virus infection and/or subjects who have received a SARS-CoV-2 vaccine. A SARS-CoV-2 virus includes wild-type SARS-CoV-2 and variants thereof including, e.g., B.1.1.7 (Alpha); B.1.351 (Beta); B.1.617.2, B.1.617.3 (Delta) and the lineages and sub-lineages designated Delta (AY.1, AY.2, and AY.3); P.1 (Gamma); B.1.525 (Eta); B.1.526 (Iota); B.1.427, B.1.429, B.1.617.1 (Kappa); B.1.621, B.1.621.1 (Mu); P.2 (Zeta); and B.1.1.529, BA.1, BA.1.1, BA.2, BA.3, BA.4 and BA.5 lineages (Omicron).

A vaccine refers to a pharmaceutical composition that elicits a prophylactic or therapeutic immune response in a subject. In some cases, the immune response is a protective immune response. Typically, vaccines elicit antigen-specific immune responses against antigens of pathogens, such as viral pathogens, or cellular components associated with pathological conditions. A vaccine can include a polynucleotide (e.g., a nucleic acid encoding a known antigen), a peptide or polypeptide (e.g., a disclosed antigen), a virus, a cell, or one or more cellular components.

In some aspects, a vaccine includes the SARS-CoV-2 Spike or S protein or nucleic acids encoding the same. The Spike protein of coronaviruses is considered crucial for determining host tropism and transmission capacity (Lu, et al. (2015) Trends Microbiol. 23:468-478; Wang, et al. (2016) Antiviral. Res. 133:165-177). The Spike protein used in a vaccine of this invention can be any wild-type or variant SARS-CoV-2 Spike protein including, but not limited to, those available under UniProtKB Accession Nos. PODTC2 and GENBANK Accession Nos. QII57278.1, YP_009724390.1, QI004367.1, QHU79173.2, QII87830.1, QIA98583.1, QIA20044.1, QIK50427. 1, QHR84449.1, QIQ08810.1, QIJ96493.1, QIC53204.1, QHZ00379.1, QHS34546.1, as well as immunogenic or antigenic fragments thereof. In certain aspects, a Spike protein used in a vaccine of this invention includes the sequence YVSQPFLMD (SEQ ID NO: 1).

A subject of this invention is an animal, preferably a mammal, in particular a human. More preferably, the subject has at least a DPB1*04:01 or DPB1*04:02 HLA allele. In the present invention, “HLA” or “human leukocyte antigen” refers to a human gene encoding Major Histocompatibility Complex (MHC) proteins on the cell surface responsible for regulating the immune system. The presence of such alleles can be readily determined by conventional methods.

In accordance with the methods herein, the presence or quantity of the T cell receptor α motif of SEQ ID NO:2 is detected in a population of immune cells from a subject. Preferably, the population of immune cells is a sample that comprises one or more antigen-specific T cells, which is obtained from a subject and is suitable for use in a subject diagnostic or monitoring assay. A “population of immune cells” encompasses blood and other liquid samples of biological origin including fine needle lymph node aspirates, solid tissue samples such as a biopsy specimen of lymphoid tissue or tissue cultures or cells derived therefrom and the progeny thereof. The definition also includes samples that have been manipulated in any way after their procurement, such as by treatment with reagents (e.g., heparinization of blood samples); washed; or enrichment for CD4⁺ T lymphocytes. The phrase “population of immune cells” encompasses a clinical sample, and includes cells in culture, tissue samples, organs, and the like. The phrase “population of immune cells” also includes preserved samples, including cryopreserved tissues, cryopreserved cell samples, and the like. In certain aspects, the population of immune cells is obtained or isolated from the lymph node of the subject.

In some aspects, the population of immune cells is enriched for CD4⁺ T cells or is a population of isolated CD4⁺ T cells. Standard methods for enriching for or isolating CD4⁺ T cells are well known in the art. For example, the methods may involve collecting the population of CD4⁺ T cells present in the sample by using a binding partner directed against a specific surface marker of the CD4⁺ T cells (e.g., a CD4 polypeptide). In a particular aspect, the method may involve bringing the sample into contact with a binding partner capable of selectively interacting with CD4⁺ T cells present in said sample. The binding partner may be an antibody that may be polyclonal or monoclonal, preferably monoclonal, directed against the specific surface marker of the CD4⁺ T cells. Typically said surface marker is CD4. In another embodiment, the binding partner may be an aptamer.

Methods of flow cytometry are preferred methods for isolating or enriching for CD4⁺ T cells in a sample. For example, fluorescence activated cell sorting (FACS) may be used to separate the desired CD4⁺ T cells. In another aspect, magnetic beads may be used to isolate CD4⁺ T cells (MACS). For instance, beads labelled with monoclonal specific antibodies may be used for the positive selection of CD4⁺ T cells. Other methods can include the isolation of CD4⁺ T cells by depletion of the cells that are not of interest (negative selection).

As demonstrated herein, the presence or quantity of a T cell receptor α motif having the amino acid sequence of SEQ ID NO: 2 in a population of immune cells is indicative of a CD4⁺ T cell response to SARS-CoV-2 infection or vaccination. For the purposes of this invention, the T cell receptor α motif is represented using the three-letter amino acid code: Cys-Ala-Xaa₁-Xaa₂-Asn-Tyr-Gly-Gly-Ser-Gln-Gly-Asn-Leu-Ile-Phe (SEQ ID NO: 2), wherein X₁ is Gly, Ala, or Val, and X₂ is any amino acid residue, or the one letter amino acid code: CA[G/A/V]XNYGGSOGNLIF (SEQ ID NO:2). One or two amino acid residue variants of this motif are also within the scope of this invention. The T cell receptor α motif of SEQ ID NO:2 can be detected and/or quantified by a number of methods including, but not limited to, the use of probes specific for SEQ ID NO:2; bulk DNA- or RNA-based TCR α repertoire sequencing of a sample; single cell TCR sequencing; targeted PCR amplification of nucleic acids encoding SEQ ID NO:2; and MHC-multimer (DPB1:04 HLA loaded with the epitope) staining of immune cell samples, with and without subsequent sequencing of TCR receptors. Any of these methods can provide information about the presence and/or quantity of epitope-specific CD4⁺ memory T cells. MHC-multimer staining combined with other surface markers of T cells can also provide information about the quality of these memory T cells.

The term “quantity,” “quantifying” or “quantitating” refers to absolute or to relative quantification. Absolute quantification may be accomplished by inclusion of known concentration(s) of one or more target nucleic acids or protein and referencing the hybridization intensity of unknowns with the known target nucleic acids or protein (e.g., through generation of a standard curve). Alternatively, relative quantification can be accomplished by comparison of signals between two or more genes or proteins, or between two or more treatments to quantify the changes in intensity and, by implication, transcription level.

In some aspects, the presence or quantity of the T cell receptor α motif comprises contacting the sample with selective reagents such as probes, primers, or ligands, and thereby detecting the presence, or measuring the amount, of polypeptide or nucleic acids of interest. Contacting may be performed in any suitable device, such as a plate, microtiter dish, test tube, well, glass, column, and so forth. In specific aspects, the contacting is performed on a substrate coated with the reagent, such as a nucleic acid array or a specific ligand array. The substrate may be a solid or semi-solid substrate such as any suitable support comprising glass, plastic, nylon, paper, metal, polymers, and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

Alternatively, the T cell receptor motif may be detected by determining the presence or quantity of mRNA. Methods for determining the quantity of mRNA are well known in the art. For example, the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e.g., northern blot analysis) and/or amplification (e.g., RT-PCR). Preferably quantitative or semi-quantitative RT-PCR is preferred. Real-time quantitative or semi-quantitative RT-PCR is particularly advantageous. Other methods of Amplification include ligase chain reaction (LCR), transcription-mediated amplification (TMA), strand displacement amplification (SDA) and nucleic acid sequence-based amplification (NASBA). Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more 90-95% preferably identical. In certain embodiments, it will be advantageous to use nucleic acids in combination with appropriate means, such as a detectable label, for detecting hybridization. A wide variety of appropriate indicators are known in the art including, fluorescent, radioactive, enzymatic, or other ligands (e.g., avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly, or almost perfectly, match a nucleic acid of interest to be amplified. The probes and primers are “specific” to the nucleic acids they hybridize to, i.e., they preferably hybridize under high stringency hybridization conditions (corresponding to the highest melting temperature Tm, e.g., 50% formamide, 5× or 6XSCC. SCC is a 0.15 M NaCl, 0.015 M Na-citrate). The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

Detection at the protein level typically comprises contacting the population of immune cells with a binding partner capable of selectively interacting with the T cell receptor α motif of SEQ ID NO:2. The binding partner is generally an antibody that may be polyclonal or monoclonal (preferably a monoclonal antibody), or an aptamer. Methods to measure protein expression levels include, but are not limited to: western blot, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of the protein including but not limited to epitope binding or interaction with other protein partners.

Nucleic acid-based TCR α repertoire sequencing may also be used in the methods of this invention. Next Generation Sequencing (NGS) or Third Generation Sequencing methods are of particular use in this invention. For clarification, the term “Next Generation Sequencing” or “NGS” in the context of the present invention means all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) can deliver nucleic acid sequence information of a whole genome, exome, transcriptome (all transcribed sequences of a genome) or methylome (all methylated sequences of a genome) in very short time periods, e.g., within 1-2 weeks, preferably within 1-7 days, or most preferably within less than 24 hours and allow, in principle, single cell sequencing approaches. Multiple NGS platforms which are commercially available or which are mentioned in the literature can be used in the context of the present invention, e.g., those described in detail in Zhang, et al. (2011) J. Genet. Genomics 38(3):95-109; Voelkerding, et al. (2009) Clin. Chem. 55:641-658.

Non-limiting examples of such NGS technologies/platforms including sequencing-by-synthesis technologies, sequencing-by-ligation, and single-molecule sequencing. A sequencing-by-synthesis technology known as pyrosequencing (Ronaghi, et al. (1998) Science 281(5375):363-365) uses an emulsion PGR in which single-stranded DNA binding beads are encapsulated by vigorous vortexing into aqueous micelles containing reactants surrounded by oil for emulsion PCR amplification. During the pyrosequencing process, light emitted from phosphate molecules during nucleotide incorporation is recorded as the polymerase synthesizes the DNA strand. The sequencing-by-synthesis approaches developed by Solexa, are based on reversible dye-terminators. In this technology, all four nucleotides are added simultaneously into oligo-primed cluster fragments in flow-cell channels along with DNA polymerase. Bridge amplification extends cluster strands with all four fluorescently labeled nucleotides for sequencing.

By comparison, in sequencing-by-ligation approaches, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. Non-limiting examples for this approach include the SOLid™ platform of Applied Biosystems and Polonator™ G. 007 platform of Dover Systems.

Single-molecule sequencing technologies provide the sequence of single DNA or RNA molecules without amplification. For example, the HeliScope™ platform (Helicos Biosciences) uses a highly sensitive fluorescence detection system to directly detect each nucleotide as it is synthesized. A similar approach based on fluorescence resonance energy transfer (FRET) has been developed from Visigen Biotechnology. Other fluorescence-based single-molecule techniques are from U.S. Genomics (GeneEngine™) and Genovoxx (AnyGene™). Electron microscopy-based technologies for single-molecule sequencing have also been described by LightSpeed Genomics and Halcyon Molecular.

Nano-technologies for single-molecule sequencing have also been described. In these approaches, various nanostructures are used which are, e.g., arranged on a chip to monitor the movement of a polymerase molecule on a single strand during replication. Non-limiting examples for approaches based on nano-technologies are the GridON™ platform of Oxford Nanopore Technologies, the hybridization-assisted nano-pore sequencing (HANS™) platforms developed by Nabsys, and the proprietary ligase-based DNA sequencing platform with DNA nanoball (DNB) technology called combinatorial probe-anchor ligation (cPAL™).

Ion semiconductor sequencing is based on the detection of hydrogen ions that are released during the polymerization of DNA. For example, Ion Torrent Systems uses a high-density array of micro-machined wells to perform this biochemical process in a massively parallel way. Each well holds a different DNA template. Beneath the wells is an ion-sensitive layer and beneath that a proprietary Ion sensor.

Preferably, DNA or RNA preparations serve as starting material for NGS. Such nucleic acids can be easily obtained from samples such as biological material, e.g., from fresh or flash-frozen tissues or from freshly isolated cells. Although nucleic acids extracted from tissues or freshly isolated single cells may be highly fragmented, they are suitable for NGS applications.

In some aspects, it is determined whether the population of CD4⁺ T cells is specific for the T cell receptor motif described herein, i.e., it is determined whether a T cell receptor motif-specific subpopulation of CD4⁺ T cells exists in the general population of immune cells. In accordance with this aspect, the epitope or peptide including the epitope of SEQ ID NO:1 is loaded on MHC class II tetramers, and the isolated antigen-induced CD4⁺ T cells are brought into contact with said tetramers. Tetramers assays are well-known in the art. To produce tetramers, the carboxyl terminus of an MHC molecule is associated with a specific peptide epitope or polyepitope and treated to form a tetramer complex having bound hereto a suitable reporter molecule, preferably a fluorochrome such as, for example, fluorescein isothiocyanate (FITC), phycoerythrin, phycocyanin or allophycocyanin. The tetramers produced bind to the distinct set of CD4⁺ T cell receptors on a subset of CD4⁺ T cells to which the peptide is MHCII restricted. Following binding, and washing of the T cells to remove unbound or non-specifically bound tetramer, the number of CD4⁺ cells binding specifically to the HLA-peptide tetramer may be quantified by standard flow cytometry methods, such as, for example, using a FACSCalibur Flow cytometer (Becton Dickinson). The tetramers can also be attached to paramagnetic particles or magnetic beads to facilitate removal of non-specifically bound reporter and cell sorting. Such particles are readily available from commercial sources (e.g., Beckman Coulter, Inc., San Diego, CA). Tetramer staining does not kill the labeled cells; therefore, cell integrity is maintained for further analysis.

The methods of this invention are useful in a wide variety of diagnostic assays, clinical studies (including preclinical studies), and monitoring assays. In some aspects, the methods may be useful for determining whether a subject has developed a memory T cell response in a context of a SARS-CoV-2 infection or conventional vaccine. In other aspects, the methods are useful for clinical studies, e.g., to compare the level of antigen-induced memory CD4⁺ T cells from one individual to another, e.g., in response to vaccination, in the absence of vaccination, etc. Accordingly, in some aspects, this invention also provides a method for determining efficacy of a vaccine for the prophylactic treatment, prevention, or amelioration of symptoms of a SARS-CoV-2 infection by detecting the presence or quantity of a T cell receptor α motif having the amino acid sequence of SEQ ID NO: 2. For example, the level of antigen-induced CD4⁺ memory T cells specific for an epitope of SEQ ID NO:1 of a vaccine preparation, in a sample from a subject who has received the vaccine is compared to the level of antigen-induced memory CD4⁺ memory T cells specific for the same antigen in a biological sample from a subject individual who has received a placebo. When the level determined in the subject administered with the vaccine preparation is higher than the level determined with the subject administered with the placebo, this indicates that the vaccine preparation was efficient. When the level determined in the subject administered with the vaccine preparation is the same or less than the level determined with the subject administered with the placebo, this indicates that the vaccine preparation had no or low efficacy. The method is particularly suitable for screening combinations of antigens and/or immunoadjuvants for the preparation of vaccine preparation. When shown to be efficacious, the vaccine preparation can subsequently be administered to a subject in need of treatment. When efficacy is not shown, one or more antigens and/or immunoadjuvants may be replaced with an improved antigen and/or immunoadjuvant to enhance efficacy.

The invention further provides methods for developing personalized treatment plans by detecting the presence and/or quantity of the TCRα chain sequence of SEQ ID NO:2. Information gained by way of the methods described above can be used to develop a personalized treatment plan for a subject (for example, a vaccinated or an immunodeficient subject). The methods can be carried out by, for example, using any of the methods of analysis described above and, in consideration of the results obtained, designing a treatment plan or a clinical course of action for the subject. If the levels of antigen-induced memory CD4⁺ memory T cells indicate that the subject has low levels of antigen-induced memory CD4⁺ memory T cells, the subject is a candidate for vaccination and/or treatment with an effective amount of immuno-stimulating agent. Depending on the level of antigen-induced memory CD4⁺ memory T cells, the recipient may require a treatment regime that is more or less aggressive than a standard regime, or it may be determined that the recipient is best suited for a standard regime. When so treated, one can treat or prevent complications associated with poor immune response. Conversely, a different result may indicate that the subject has high levels of antigen-induced memory CD4⁺ memory T cells and/or shows immune protection and is not likely to experience an undesirable clinical outcome (e.g., being at risk of infection). In that event, the patient may avoid vaccination and/or treatment with immuno-stimulating agents (or require a less aggressive regime) and their associated side effects.

The invention also features kits for determining whether a subject exhibits a CD4⁺ memory T cell response, assessing the efficacy of a vaccine or a treatment for inducing/maintaining CD4⁺ memory T cell response and/or a protective immune response in a subject. The kits can include reagents for evaluating the presence or quantity of a T cell receptor α motif having the amino acid sequence of SEQ ID NO: 2 or nucleic acids (e.g., mRNAs) encoding the same. Kits for evaluating expression of nucleic acids can include, for example, probes or primers that specifically bind a nucleic acid of interest (e.g., a nucleic acid, the expression of which correlates with the presence or absence a T cell receptor α motif having the amino acid sequence of SEQ ID NO: 2 in a sample). The kits for evaluating nucleic acid expression can provide substances useful as standard (e.g., a sample containing a known quantity of a nucleic acid to which test results can be compared, with which one can assess factors that may alter the readout of a diagnostic test, such as variations in an enzyme activity or binding conditions). Kits for assessing nucleic acid expression can further include other reagents useful in assessing levels of expression of a nucleic acid (e.g., buffers and other reagents for performing PCR reactions, or for detecting binding of a probe to a nucleic acid). In addition to, or as an alternative, kits can include reagents (e.g., antibodies) for detecting proteins. The kits can provide instructions for performing the assay used to evaluate gene or protein expression, and instructions for determining immune response, efficacy and/or risk based on the results of the assay. For example, the instructions can indicate that levels of expression of a T cell receptor α motif having the amino acid sequence of SEQ ID NO: 2 or nucleic acid encoding the same (e.g., relative to a standard or a control), correlate with the presence or absence of SARS-CoV-2 Spike protein-specific CD4⁺ memory T cells. Kits can also provide instructions, containers, and other reagents for obtaining and processing samples for analysis.

The invention is described in greater detail by the following non-limiting examples.

### Example 1: Materials and Methods

Human Subjects. Human subjects who elected to receive the BNT162b2 mRNA vaccine were recruited into this prospective observational study. Written informed consent was obtained from each subject. The study was approved by the Washington University in St. Louis Institutional Review Board (approval #2020-12-081). Details of the entire study cohort have been previously reported (Turner et al. (2021) Nature 596:109-113). Draining axillary lymph nodes ipsilateral to the deltoid vaccination site were located with ultrasound and sampled with multiple passes of 6 separate 25-gauge needles under real-time ultrasound guidance (Turner et al. (2020) Nature 586:127-132). Each needle was flushed with 3 mL of R10 (RPMI, 1640 supplemented with 10% FBS and 100 U/mL penicillin-streptomycin) and the three separate 1 mL rinses of R10. Red blood cells were lysed with 1xACK (Sacha & Watkins (2010) Nat. Protoc. 5:239-246) and then washed with P2 (1xPBS supplemented with 2% FBS and 2 mM EDTA). FNA samples were immediately stained for flow cytometry or cryopreserved in freezing media (10% dimethyl sulfoxide and 90% FBS). Matched blood samples from the same time-points were obtained by standard phlebotomy into EDTA anti-coagulated tubes and PBMC were prepared by density gradient centrifugation over a copolymer of sucrose and epichlorohydrin sold under the tradename FICOLL® 1077 (GE). PBMC were treated with 1xACK for 5 minutes to lyse residual red blood cells before washing with R10 and immediate use in flow cytometry experiments or cryopreservation in freezing media.

For S₁₆₇₋₁₈₀ tetramer validation and ICS experiments PBMC were used from SARS-CoV-2 convalescent and vaccinated donors obtained as a part of the St. Jude Tracking of Viral and Host Factors Associated with COVID-19 study (SJTRC, NCT04362995); a prospective, IRB-approved, longitudinal cohort study of St. Jude Children's Research Hospital adult (R18 years old) employees. Participants were screened for SARS-CoV-2 infection by PCR approximately weekly when on St. Jude campus. For this study, the convalescent blood draw for SARS-CoV-2 infected individuals (3-8 weeks post diagnosis) as well as post-vaccination blood draws for SARS-CoV-2 naïve individuals were used. Blood samples were collected in 8 mL CPT tubes; and PBMC were isolated and frozen within 24 hours of collection. HLA typing of each included SJTRC participant was performed using the AllType NGS 11-Loci Amplification Kit (One Lambda) according to manufacturer's instructions. Resulting libraries were sequenced on MiSeq lane at 150×150 bp. HLA types were called using the TypeStream Visual Software from One Lambda.

Cell Sorting and Flow Cytometry. Fresh or frozen PBMC and/or FNA samples were washed and re-suspended in P2. For sorting of THE populations from frozen FNA samples, cells were stained with ALEXA FLUOR® 700 (fluorescent dye) labeled anti-. CD4 antibody (SK3, anti-CD19 BioLegend), PE (HIB19, BioLegend), anti-CXCR5 PE-DAZZLE™ 594 (J252D4, BioLegend), anti-PD1 BB515 (EH12.1, BD Horizon), and ZOMBIE AQUA™ (BioLegend) for a total of 30 minutes on ice. Cells were then washed twice with P2 and live, singlet, CD4⁺CD19-CXCR5⁺PD1⁺ cells were sorted on a FACSAria II into a monophasic solution of phenol and guanidine isothiocyanate sold under the tradename TRIZOL® before being immediately frozen on dry ice.

Jurkat Cell Line Generation. For Jurkat cell line generation a TCRα (TRAV35, CAGMNYGGSOGNLIF, TRAJ42, SEQ ID NO: 3) and two different TCRβ chains (TRBV4-1, CASSQGVGYTF, TRBJ1-2, SEQ ID NO: 4; TRBV6-3, CASSYRGAYGYTF, TRBJ1-2, SEQ ID NO: 5) were selected from Bacher et al. ((2020) Immunity 53:1258-1271.e5). Both TCRα and TCRβ chains were modified to use murine constant regions to facilitate surface expression (murine TRAC*01 and murine TRBC2*01). Two gBlock gene fragments were synthesized by Genscript to encode the modified TCRα chain, one of the modified TCRβ chains, and mCherry fluorescent protein, linked together by 2A sites. These sequences were cloned into the pLVX-EF1α-IRES-Puro lentiviral expression vector (Clontech). To generate the lentivirus, 293T packaging cell line (ATCC CRL-3216) was transfected with the pLVX lentiviral vector containing TCR_4.1-mCherry or TCR_6.3-mCherry insert, psPAX₂ packaging plasmid (Addgene plasmid #12260), and pMD2.G envelope plasmid (Addgene plasmid #12259). Viral supernatant was collected and concentrated using Lenti-X Concentrator 24- and 48-hours after the transfection (Clontech). Jurkat 76.7 cells (variant of TCR-null Jurkat 76.7 cells that expresses human CD8 and an NFAT-GFP reporter) were transduced, then antibiotic selected for 1 week using 1 mg/mL puromycin in RPMI (Gibco) containing 10% FBS and 1% penicillin/streptomycin. Transduction of Jurkat cell line was confirmed by expression of mCherry, and surface TCR expression was confirmed via flow cytometry on a BD Fortessa using FACSDiva software using antibodies against mouse TCRβ constant region (APC-Fire750-conjugated, Biolegend, clone H57-597) and human CD3 (Brilliant Violet 421-conjugated, Biolegend, clone SK7). Flow data were analyzed in FlowJo software.

Jurkat Peptide Stimulation. Jurkat 76.7 cells expressing TCRs 4.1 and 6.3 (2.5×10⁵) were co-cultured with PBMCs from SARS-CoV-2 naïve DPB1*:04:01-positive donor (6×10⁵) pulsed with 1 μM of peptide, 1 μg/mL each of anti-human CD28 and CD49d (BD Biosciences). An unstimulated (CD28, CD49d) and positive control (CD28, CD49d, 1× Cell Stimulation Cocktail, PMA/ionomycin; eBioscience) were included in each assay. Cells were incubated for 18 hours (37° C., 5% CO₂). After the incubation cells were washed twice with FACS buffer (PBS, 2% FBS, 1 mM EDTA), resuspended in 50 μL of FACS buffer, and then blocked using 1 μL human Fc-block (BD Biosciences). Cells were then stained with 1 μL GHOST DYE™ Violet 510 Viability Dye (Tonbo Biosciences) and a cocktail of fluorescent antibodies: 1 μL each of anti-human CD3 (Brilliant Violet 421-conjugated, Biolegend, clone SK7), anti-human CD69 (PerCP-eFluor710-conjugated, eBioscience, clone FN50), and anti-mouse TCRβ chain (APC/Fire750-conjugated, Biolegend, clone H57-597). Cells were incubated for 20 minutes at room temperature and then washed with a FACS buffer. Cells were analyzed by flow cytometry on a custom-configured BD Fortessa using FACSDiva software (Becton Dickinson). Flow cytometry data were analyzed using FlowJo software (BD Biosciences). Responsiveness to peptide stimulation was determined by measuring frequency of NFAT-GFP, CD69 and αβTCR expression.

Monomer Generation. HLA-DP4 monomers with the S₁₆₇₋₁₈₀ epitope were produced from purified HLA-DP4 containing the class II-associated invariant chain peptide (CLIP) (Niehrs et al. (2019) Nat. Immunol. 20:1129-37) via HLA-DM-catalyzed peptide exchange as described previously for HLA-DR (Scally et al. (2013) J. Exp. Med. 210:2569-2582). Briefly, HLA-DP4 CLIP was expressed in Trichoplusia ni (Hi5) insect cells via a pFastBac-Dual construct encoding HLA-DPA1*01:03 α- and HLA-DPB1*04:01 β-chains with C-terminal fos/jun zipper domain. The HLA-DP4 β-chain further contained an N-terminal factor Xa cleavable CLIP sequence, and a C-terminal biotinylation signal and His₇ tag (Niehrs et al. (2019) Nat. Immunol. 20:1129-37). Following expression for 3 days at 27° C., cell supernatant was concentrated and buffer exchanged Tangential Flow Filtration system into 500 mM NaCl, 10 mM Tris-HCl pH 8 and subsequently purified via immobilized metal affinity chromatography and SUPERDEX® S200 gel permeation chromatography (GPC) in 150 mM NaCl, 10 mM Tris-HCl pH 8. The linked CLIP peptide was cleaved with factor Xa for 6 hours at 21° C. prior to peptide exchange, and factor Xa cleaved HLA-DP4 was subsequently incubated in the presence of a 10-fold molar excess of peptide and a 1/5 molar ratio of HLA-DM for 16 hours at 37° C. in 100 mM sodium citrate pH 5.4. HLA-DP4 loaded with S₁₆₇₋₁₈₀ peptide was buffer-exchanged into 50 mM NaCl, 20 mM Tris-HCl pH8, purified via HI-TRAP® Q ion exchange chromatography and biotinylated using BirA biotin ligase. Following a final SUPERDEX® S200 GPC step in PBS, biotinylated HLA-DP4-S₁₆₇₋₁₈₀ monomer was concentrated to approximately 1 mg/ml and stored at −80° C.

Tetramer Generation and Staining of Jurkat Cells. Biotinylated HLA-DP4-monomers loaded with TFEYVSOPFLMDLE peptide (S₁₆₇₋₁₈₀, SEQ ID NO: 6) were tetramerized using PE-Streptavidin (Biolegend). One volume PE-conjugated streptavidin was added to one volume of HLA-DP4-monomer (1 mg/mL). The volume of PE-streptavidin (0.2 mg/ml) was divided in four parts and added in four consecutive steps with 10 minutes incubation between. After adding all needed amounts of PE-streptavidin the mixture was incubated for at least 1 hour on ice prior to staining. Jurkat 76.7 cells expressing TCR4. 1, TCR6. 3, Jurkat 76.7 cell line expressing irrelevant TCR (specific to NOKLIANQF (SEQ ID NO:7) epitope from the spike protein of SARS-CoV-2 (Minervina et al. (2021) Nat. Immunol. 23:781-90), and SARS-CoV-2 naïve HLA-DPB1*04:01 positive donors' PBMCs were stained with 1 μL GHOST DYE™ Violet 510 Viability Dye (Tonbo Biosciences) and 1 μL of HLA-DPB1*04-S167-180-tetramer. Cells were analyzed by flow cytometry on a custom-configured BD Fortessa using FACSDiva software (Becton Dickinson). Flow cytometry data were analyzed using FlowJo software (BD Biosciences). The quality of the S₁₆₇₋₁₈₀ HLA class II tetramer was judged by staining of the relevant T cell line and low background in irrelevant Jurkat cells and naïve PBMCs.

Bulk Repertoire Generation. TCRalpha and TCRbeta bulk repertoires were generated with an established 5′ RACE protocol (Egorov et al. (2015) J. Immunol. 194:6155-6163). RNA was isolated using a monophasic solution of phenol and guanidine isothiocyanate sold under the tradename TRIZOL® (Invitrogen) according to the manufacturer's protocol. All RNA was used for cDNA synthesis with SMARTScribe™ kit (Takara), using template switch oligonucleotide and primers specific for TCRalpha and TCRbeta constant segments. cDNA was amplified in two rounds of PCR using Q5 high-fidelity polymerase (NEB). Adapters necessary for sequencing on the Illumina platform were introduced with the KAPA HyperPrep kit (Roche). Libraries were sequenced on Illumina MiSeq platform.

Public TCR Repertoire Datasets. TCRbeta dataset for MIRA class II peptide stimulation (ImmuneCODE MIRA release 002.1) was accessed via ImmuneACCESS database (Nolan et al. (2020) Res. Sq. 1:51964). Processed single cell paired chain TCR datasets from ARTE assays after 6 and 24 hour stimulation with SARS-CoV-2 peptides were used as supplied by authors in original publications: Table S3 from Bacher et al. ((2020) Immunity 53:1258-1271.e5) and Table S4A from Meckiff et al. ((2020) Cell 183:1340-1353.e16).

TCR Repertoire Analysis. Bulk TCR repertoire data was demultiplexed and assembled into the UMI consensuses with migec (v. 1.2.7; with collision filter and force-overseq parameters set to 1) (Shugay et al. (2014) Nat. Methods 11:653-655). V and J-segment alignment, CDR3 identification and assembly of reads into clonotypes were performed with MiXCR (v. 3.0.3) with default parameters (Bolotin et al. (2015) Nat. Methods 12:380-381). Resulting processed repertoire datasets and reference to raw TCR repertoire sequencing data are available at GEO database (acc. GSE183393). Analysis of bulk repertoire data was performed using R language for statistical computing, with merging and subsetting of data performed using data.table package. stringdist and igraph R packages were used to build TCR similarity network, gephi software was used for TCR similarity networks layout and visualization and ggplot2 library for other visualizations.

### Example 2: SARS-CoV-2 Spike Peptide Elicits Robust T Follicular Helper Cell Responses

To characterize antigen responses of CD4⁺ T_{FH} cells, total T_{FH} cell populations from FNA samples obtained on day 60 from four donors were sorted and their T cell repertoires were reconstructed. While a prominent TCRβ chain repertoire was not observed, clonally expanded TCR clonotypes formed a prominent TCRα chain cluster that was shared among all four donors. A highly conserved TCR motif of this cluster, characterized by a TRAV35-CA[G/A/V]XNYGGSOGNLIF-TRAJ42 (SEQ ID NO: 2), has also been observed in 0.2% of the total CD4⁺ cells and 16.3% of estimated SARS-CoV-2-responding CD4⁺ cells in the blood at the peak of the acute response to the covid infection (Minervina, et al. (2021) eLife 10:e63502). Such big clusters of TCRs with similar sequences are a sign of convergent selection of similar receptors to the same antigen (Dash, et al. (2017) Nature 547:89-93; Glanville, et al. (2017) Nature 547:94-98; Pogorelyy, et al. (2019) PLoS Biol. 17:e3000314). As this motif was present among expanded clones in many donors it likely recognizes an immunodominant epitope from SARS-CoV-2 presented on the common HLA class II.

To decode the specificity of the αβTCR heterodimer, the β chains corresponding to the TCRα chain motifs were determined. This analysis was carried out by analyzing publicly available CD4⁺-paired TCR datasets. Notably, there were two datasets that had paired αβTCRs from CD4⁺ cells after stimulation with SARS-CoV-2 peptides and antigen-reactive T cell-enrichment assay (Meckiff, et al. (2020) Cell 183:1340-1353.e16; Bacher, et al. (2020) Immunity 53:1258-1271.e5). These datasets were searched using the CDR3α motif (CA[G/A/V]XNYGGSQGNLIF; SEQ ID NO: 2) and 1329 unique TCRs were found in the dataset of Bacher, et al. ((2020) Immunity 53:1258-1271.e5). By comparison, only 53 unique TCRs were found in the dataset of Meckiff, et al. ((2020) Cell 183:1340-1353.e16). Identified β chains were subsequently used to search for overlap in the MIRA™ dataset produced by Adaptive Biotech, the largest dataset linking TCR sequences to SARS-CoV-2 epitopes (Nolan, et al. (2020) Res. Sq. 1:51964). This analysis identified 64 TCRs from Bacher, et al. ((2020) Immunity 53:1258-1271.e5) that were highly similar (up to one amino acid mismatch in CDR3 with the same CDR1 and CDR2 sequences) to MIRA™ TCRs reactive to the overlapping peptide pool from SARS-CoV-2 Spike protein 160-218 positions (S₁₆₀₋₂₁₈). Notably, this part of the Spike protein was not used for stimulation in Meckiff, et al. ((2020) Cell 183:1340-1353.e16), explaining why only a few TCRs of interest were found in this dataset.

Out of six subjects from the MIRA™ database, five were HLA-typed and shared DPB1*04:(01/02) and DQB1*06:(02/03) alleles. To establish HLA-restriction and narrow the search to single peptides, the NetMHCII2. 3 prediction method (Jensen, et al. (2018) Immunology 154:394-406) was used determine the predicted epitopes from the S₁₆₀₋₂₁₈ peptide pool presented by these shared alleles.

Peptides containing the core sequence YVSQPFLMD (SEQ ID NO: 1) were predicted to bind strongly to DPB1:04:01 and DPB1:04:02 alleles, while no strong binders were identified for DQB1*06:(02/03) alleles. Of note, a TCR epitope with the core sequence CTFEYVSQPFLMDLE (S₁₆₆₋₁₈₀) (SEQ ID NO: 8) has been found in epitope discovery studies (Peng, et al. (2020) Nat. Immunol. 21:1336-1345; Tarke, et al. (2021) Cell Rep. Med. 2:100204). In these studies, the response to this peptide was identified in multiple donors, but it was not predicted to be restricted to DPB1*04.

Having identified a paired TCR, the peptide epitope and the restricting HLA, the recognition of the epitope was experimentally determined. This analysis was carried out by selecting two paired TCRs of Bacher, et al. ((2020) Immunity 53:1258-1271.e5), which had the same TCRα but different β chains, and transducing the same into a Jurkat TCR-null cell line. Recognition of the epitope was subsequently confirmed in a peptide stimulation assay with peripheral blood mononuclear cells (PBMCs) from DPB1*04-positive donors used as antigen presenting cells (APCs).

A Major histocompatibility complex (MHC)-tetramer was subsequently generated to probe antigen-specific T cell responses ex vivo. The tetramer was tested using Jurkat cell lines with known specificity, high sensitivity, and low-background levels. Using the S₁₆₆₋₁₈₀ (CTFEYVSQPFLMDLE) (SEQ ID NO: 8) tetramer, PBMCs from three SARS-CoV-2 convalescent donors or SARS-CoV-2-naïve donor controls were screened for antigen-specific CD4⁺ T cells. Tetramer-specific cells were predominantly found in a naïve subpopulation (CCR7⁺CD45RA⁺) in the naïve donor, and in the effector memory subpopulation (CCR7⁻CD45RA⁻) in the SARS-CoV-2 convalescent donors. Tetramer-specific TCRs were subsequently sequenced using a scTCRseq approach (Wang, et al. (2012) Sci. Transl. Med. 4:128ra42). This analysis indicated that the majority (64%) of the TCRs had the same TRAV35-CA[G/A/V]NYGGSQGNLIF (SEQ ID NO: 2) TCRα motif, with >80% of all sequences having TRAV35. Thus, this motif is the most frequent mode of recognition for this epitope.

1. A method for determining whether a subject exhibits a CD4⁺ memory T cell response to SARS-CoV-2 infection or vaccination comprising detecting, in a population of immune cells from the subject, the presence or quantity of a T cell receptor α motif having the amino acid sequence Cys-Ala-Xaa₁-Xaa₂-Asn-Tyr-Gly-Gly-Ser-Gln-Gly-Asn-Leu-Ile-Phe (SEQ ID NO: 2), wherein X₁ is Gly, Ala, or Val, and X₂ is any amino acid residue, wherein the presence or quantity of the T cell receptor α motif indicates that the subject exhibits a CD4⁺ memory T cell response to SARS-CoV-2 infection or vaccination.

2. The method of claim 1, wherein the subject has at least a DPB1*04:01 or DPB1*04:02 HLA allele.

3. A method for determining efficacy of a SARS-CoV-2 vaccine comprising detecting, in a population of immune cells from a subject vaccinated with the SARS-CoV-2 vaccine, the presence or quantity of a T cell receptor α motif having the amino acid sequence Cys-Ala-Xaa₁-Xaa₂-Asn-Tyr-Gly-Gly-Ser-Gln-Gly-Asn-Leu-Ile-Phe (SEQ ID NO: 2), wherein X₁ is Gly, Ala, or Val, and X₂ is any amino acid residue, wherein the presence or quantity of the T cell receptor α motif is indicative of the efficacy of the SARS-CoV-2 vaccine.

4. The method of claim 3, wherein the subject has at least a DPB1*04:01 or DPB1*04:02 HLA allele.

5. A method for developing a personalized SARS-CoV-2 treatment plan for a subject comprising detecting, in a population of immune cells from the subject, the presence or quantity of a T cell receptor α motif having the amino acid sequence Cys-Ala-Xaa₁-Xaa₂-Asn-Tyr-Gly-Gly-Ser-Gln-Gly-Asn-Leu-Ile-Phe (SEQ ID NO: 2), wherein X₁ is Gly, Ala, or Val, and X₂ is any amino acid residue, wherein the presence or quantity of the T cell receptor α motif is indicative of a personalized SARS-CoV-2 treatment plan for the subject.

6. The method of claim 5, wherein the subject has at least a DPB1*04:01 or DPB1*04:02 HLA allele.


SEQUENCE LISTING (8 sequences)

SEQ ID NO: 1 - protein, 9 aa, Severe Acute Respiratory Syndrome Coronavirus 2
YVSQPFLMD

SEQ ID NO: 2 - protein, 15 aa, synthetic construct
  VARIANT 3: note = X can be G or A or V
  VARIANT 4: note = X can be any amino acid
CAXXNYGGSQGNLIF

SEQ ID NO: 3 - protein, 15 aa, Homo sapiens
CAGMNYGGSQGNLIF

SEQ ID NO: 4 - protein, 11 aa, Homo sapiens
CASSQGVGYTF

SEQ ID NO: 5 - protein, 13 aa, Homo sapiens
CASSYRGAYGYTF

SEQ ID NO: 6 - protein, 14 aa, Severe Acute Respiratory Syndrome Coronavirus 2
TFEYVSQPFLMDLE

SEQ ID NO: 7 - protein, 9 aa, Severe Acute Respiratory Syndrome Coronavirus 2
NQKLIANQF

SEQ ID NO: 8 - protein, 15 aa, Severe Acute Respiratory Syndrome Coronavirus 2
CTFEYVSQPFLMDLE